(19) United States
(12) Patent Application Publication
(10) Pub. No.: US 2013/0150733 A1
(43) Pub. Date: Jun. 13, 2013

(54) DEVICE AND METHOD FOR DETECTING AN EMBOLUS MOVING IN A BLOOD VESSEL

(71) Applicant: Sasi Solomon, Herzliya (IL)

(72) Inventor: Sasi Solomon, Herzliya (IL)

(21) Appl. No.: 13/817,592

(22) PCT Filed: Aug. 18, 2011
(86) PCT No.: PCT/IL2011/000674

§ 371 Date: Feb. 19, 2013

Related U.S. Application Data
(60) Provisional application No. 61/374,702, filed on Aug. 18, 2010.

(51) Int. Cl. A61B 5/00; A61B 5/02

(52) U.S. Cl. CPC A61B 5/0082; A61B 5/7275; A61B 5/746; A61B 5/02007
USPC 600/479

(57) ABSTRACT

The invention provides a device for detecting a moving embolism in a blood vessel. One or more sources of electromagnetic radiation deliver electromagnetic radiation to the blood vessel. First and second optical detectors, positioned at a downstream and an upstream location, respectively, along the flow axis of the blood vessel, detect electromagnetic radiation emitted by the sources at the downstream and upstream locations, after having been reflected from, or transmitted through, the blood vessel. A processor monitors one or more parameters of the electromagnetic radiation detected by the downstream and upstream detectors. An embolus in the blood vessel is detected when a transient change in a monitored parameter is first detected by the downstream detector, and, after a time lag, is then detected by the upstream detector.

### PRIORITY INFORMATION

This National Stage Application claims priority to U.S. Provisional Application No. 61/374,702 filed Aug. 18, 2010, and PCT Application No. PCT/IL2011/000674 filed Aug. 18, 2011.

### FIELD OF THE INVENTION

This invention relates to medical devices and more particularly to such devices for the detection of an embolus.

### BACKGROUND OF THE INVENTION

The term “embolus” is used to refer to any detached, itinerant intravascular mass (solid, liquid, gaseous, or gel like) as carried by circulation. The major source of emboli is a thrombus (blood clot) that has detached from a blood vessel wall or created by arterial fibrillation.

An embolus that is small enough to flow unimpeded in the arteries is capable of becoming lodged in an arterial or vain capillary and clogging the capillary. The term “embolism” refers to an event of lodging of an embolus in a narrow capillary vessel of an arterial bed which causes a blockage (vascular occlusion). The main complication of arterial embolism is infarction, that tissue death (necrosis) caused by blockage of the tissue's blood supply. Emboli often have more serious consequences when they occur in areas of the body that have no redundant blood supply, such as the brain (above the circle of Willis) and heart. When a clot reduces blood flow or blocks arteries which supply blood to the brain, a brain stroke may occur. If the condition is not quickly alleviated permanent damage might occur. Thus, the presence of an embolus moving in a blood vessel can precede transient ischemic attack (TIA), stroke and other conditions resulting from vessel occlusion.

One method for detecting an occluded blood vessel measures blood flow in a blood vessel. U.S. Pat. Nos. 5,348,015 and 6,547,736, for example, disclose determining blood flow in a blood vessel using Doppler ultrasound to detect occluded blood vessels. These methods, however, can only detect an embolism only after the embolism has occluded the blood vessel and are incapable of detecting an embolus before the occlusion has occurred.

U.S. Pat. No. 7,789,830 to Ishida et al and U.S. Pat. No. 7,771,358 to Moehring et al disclose devices for detecting an embolus moving in a blood vessel. Ultrasound radiation or light is directed through the skin at a single location in the blood vessel just below the skin surface. The reflected signal is monitored for transient perturbations in the reflected signal indicative of passage of an embolus at that location in the blood vessel, and estimating the size of the embolus from the signal. The device may be attached to the user's neck over the carotid artery.

### SUMMARY OF THE INVENTION

The present invention provides a non-invasive device for real-time and/or online monitoring of a blood vessel for the presence of an embolus moving in the blood vessel. The blood vessel may be, for example, a surface vessel, such as a carotid artery.

The device of the invention comprises one or more electromagnetic radiation emitters and two or more optical detectors positioned along the flow axis of the vessel. For a surface blood vessel, the emitters and optical detectors are positioned on the skin surface overlying the longitudinal axis of the blood vessel. In this case, electromagnetic radiation emitted by the radiation emitter is directed towards the blood vessel and is reflected from the blood vessel towards the optical detectors. The presence of an embolus moving in the blood vessel in the field of view (FOV) of the optical detectors is detected as a transient change in one or more parameters of the reflected or transmitted light detected by the upstream detector that occurs with a time lag after detection of a transient change in the parameters of the reflected or transmitted light by the downstream detector. For an artery, the downstream location is located along the circulatory system closer to the heart than the upstream location. A parameter may be, for example, the intensity of the light, and the transient change may include, for example, a peak or dip in the light intensity. The peaks or dips detected by the upstream and downstream detectors may partially overlap in time.

The invention thus provides a device for detecting a moving embolism in a first blood vessel comprising:

(a) one or more sources of electromagnetic radiation adapted to deliver electromagnetic radiation to the first blood vessel;

(b) a first optical detector detecting at a first location along the first blood vessel electromagnetic radiation produced by the one of the sources of electromagnetic radiation after the electromagnetic radiation has interacted with blood flowing in the first blood vessel;

(c) a second optical detector detecting at a second location along the first blood vessel electromagnetic radiation produced by the one of the sources of electromagnetic radiation after the electromagnetic radiation has interacted with blood flowing in the first blood vessel, the first location being downstream along the first blood vessel from the second location;

(d) a processor configured to perform the following steps:
(i) monitor one or more parameters of the electromagnetic radiation detected by the first optical detector and monitor the one or more parameters of the electromagnetic radiation detected by the second optical detector; and
(ii) detect a first predetermined or statistically significant transient change in one or more of the monitored parameters of the electromagnetic radiation detected by the first optical detector, and detect a second predetermined or statistically significant transient change in one or more of the monitored parameters of the electromagnetic radiation detected by the second optical detector, the second transient change occurring with a time lag after the occurrence of the first transient change; and
(iii) generate a sensible signal when a first predetermined or statistically significant transient change in one or more of the monitored parameters of the electromagnetic radiation is detected by the first optical detector, and a second predetermined or statistically significant transient change in one or more of the monitored parameters of the electromagnetic radiation is detected by the second optical detector, the second transient change occurring with a time lag after the occurrence of the first transient change.

A monitored parameter may be, for example, an intensity of the light. The first blood vessel may be, for example, a carotid artery. Two or more of the optical detectors may be configured to detect reflected electromagnetic radiation.

The device of the invention may further comprise an alarm and the sensible signal is a signal produced by the alarm. The device may also comprise a transceiver for communicating with a remote location. The device may comprise two or more sources of electromagnetic radiation, each of the two or more sources of electromagnetic radiation producing light having a different wavelength.

One or more sources of electromagnetic radiation and the first and second optical detectors may be contained in one or more optical assemblies located in one or more detection units, each detection unit being configured to be immobilized at a location selected from (a) a skin surface over the first blood vessel, (b) a surface on the first blood vessel, or (c) a location under the skin surface over the first blood vessel. The first and second optical detectors may be positioned in the optical assembly on opposite sides of a source of electromagnetic radiation. The detection unit may be configured to be immobilized at a location with the first and second optical detectors positioned along a longitudinal axis of the blood vessel.

The device may comprise one or more additional optical detectors in which case, the processor may be further configured to monitor one or more parameters of radiation detected by the one or more additional optical detectors. In this case, the step of detecting an embolus moving in the blood vessel may further comprise detecting a transient change in one or more of the parameters of radiation detected by one or more of the additional optical detectors. For example, the device may comprise a third optical detector and a fourth optical detector, the third and fourth optical detectors being positioned on a line transverse to a line between the first and second detectors. The optical detectors may be arranged in a matrix.

The device may further comprise an impedance detector and the step of detecting an embolus moving in the blood vessel further comprises detecting a transient change in local body impedance. The device may further comprise one or more additional detection units each detection unit being configured to be immobilized at a location selected from (a) a skin surface over a second blood vessel, (b) a surface on a second blood vessel, or (c) a location under the skin surface over a second blood vessel. The second blood vessel maybe the same as the first blood vessel or the second blood vessel may be different from the first blood vessel. The first optical detector and the second optical detector may be located in different optical assemblies.

In this case, one or both of the predetermined thresholds may be periodically updated. The step of detecting an embolus moving in the blood vessel may further comprise detecting a transient change in a spectral property of the blood.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 shows schematically a device for detecting an embolus moving in a blood vessel in accordance with one embodiment of the invention;

FIG. 2 shows an optical assembly for use in the device of FIG. 1 ;

FIG. 3a shows a side view of the detection unit of the device of FIG. 1 after having been affixed to a skin surface overlying a blood vessel; and

FIG. 3b shows a top view of the detection unit of the device of FIG. 1 after having been affixed to a skin surface overlying a blood vessel;

### DESCRIPTION OF THE INVENTION

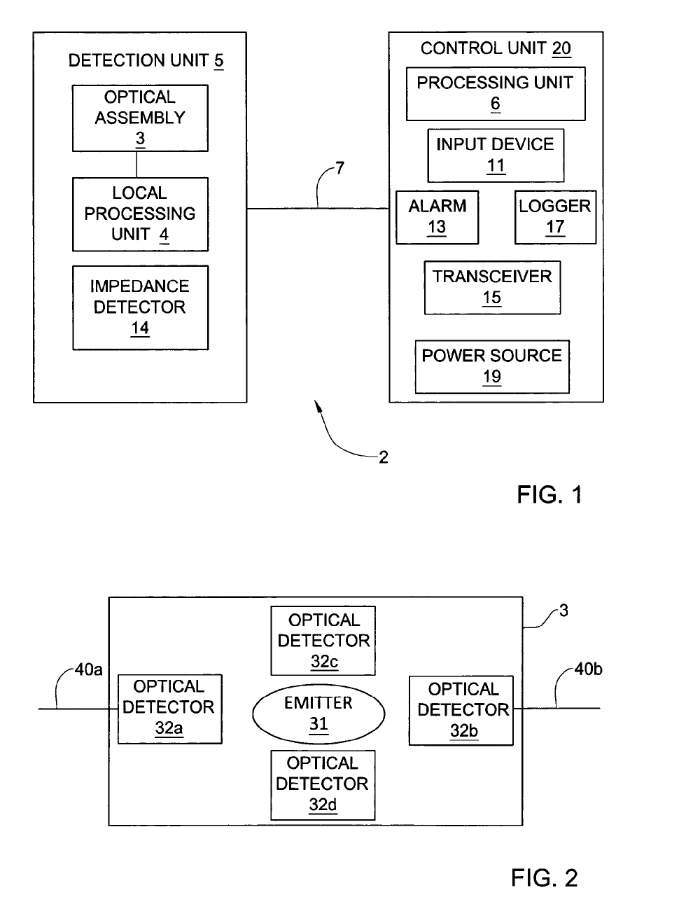

FIG. 1 shows schematically a device 2 for detecting an embolus moving in a surface blood vessel in accordance with one embodiment of the invention. The device 2 comprises a detection unit 5 and a control unit 20. The control unit 20 may be integral with the detection unit 5, or the control unit 20 may be separate from the detection unit 5 and communicate with the detection unit 5 via a wired or wireless communication link 7 The detection unit 5 is adapted to be secured to a skin surface over a blood vessel to be monitored. The control unit 20, if it is not integral with the detection unit 5, may be portable and worn by the user. For example, the control unit may be contained in a pouch attached to the user's belt. Alternatively, the control unit 5 may not be portable and placed alongside a bed ridden patient. The control unit also includes a power supply 19 that may also supply power to the detection unit 5.

The detection unit 5 includes one or more optical assemblies 3 and a local processing unit 4 and may include an impedance detector 14. The control unit 20 includes a processing unit 6. The detection unit 5 and\or the control unit 20 may include an analog to digital converter, a digital to analog converter and a memory. The control unit further comprises a user input device 11 such as a keypad that may be used to input relevant data or to configure the processing unit 6 and optical assembly 3. The control unit 20 also includes an alarm 13 that generates a sensible signal when an embolus is detected in a blood vessel, and may also include a logger 17. A transceiver 15 such as a Bluetooth transceiver or an SMS sender may be used to transmit and receive data to a remote location for further analysis or data storage.

FIG. 2 shows the optical assembly 3 in greater detail in accordance with one embodiment of the invention. The optical assembly 3 includes one or more electromagnetic radiation emitters 31. Each emitter 31 is capable of emitting electromagnetic radiation at one or more different wavelengths. Two or more of the optical detectors 32 are arranged around the one or more electromagnetic radiation emitters 31 in the optical assembly. Four optical detectors 32a, 32b, 32c, and 32d, are shown in FIG. 2. This is by way of example only, and the detection unit 5 may include any number of optical detectors that is at least two. An optical detector 32 may be, for example, a photodiode and the optical detectors 32 may be arranged in a matrix. Each optical detector 32 may be provided with one or more light filters of appropriate wavelengths.

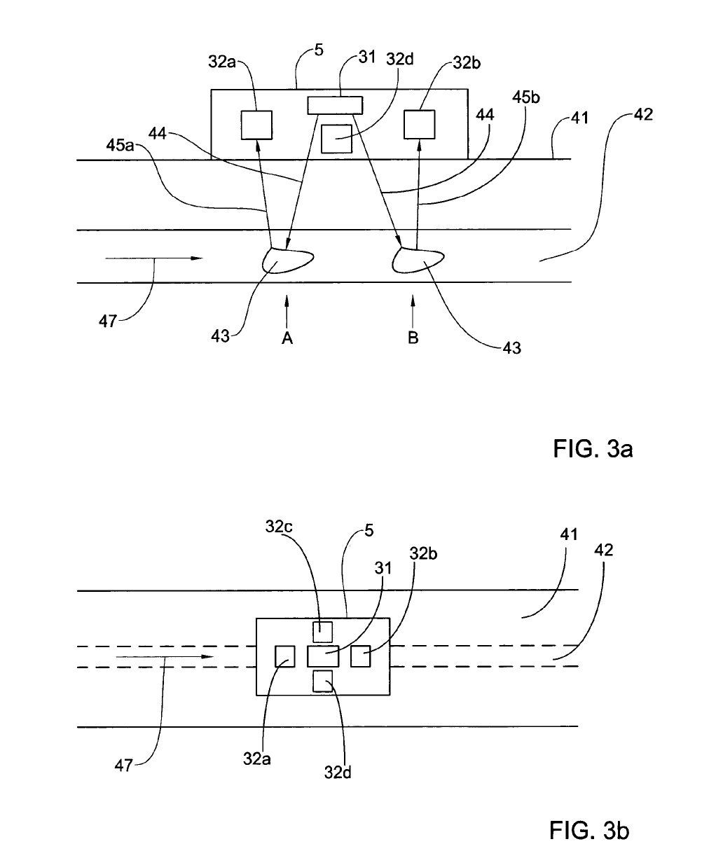

FIG. 3a shows a side view of the detection unit 5 after having been affixed to a skin surface 41 overlying a blood vessel 42, and FIG. 3b shows a top view of the detection unit 5 after having been affixed to a skin surface 41 overlying a blood vessel 42. The detection unit 5 is positioned on the skin surface 41 with the optical detectors 32a and 32b lying on the longitudinal axis of the blood vessel 42 with the emitter 31 being located between the optical detectors 32a and 32b. In the blood vessel 42, blood is flowing from left to right, as indicted by the arrow 47, so that the optical detector 32a is located downstream to the detector 32b. One or more of the detectors, such as the detectors 32c and 32d, may be positioned lateral to the radiation emitters 31.

Referring still to FIG. 3, electromagnetic radiation 44 emitted by the radiation emitter 31, is directed towards the blood vessel 42. Electromagnetic radiation 45 reflected from the blood vessel 43 is detected by the optical detectors 32. The presence of an embolus 43 in the blood vessel 42 in the FOV of the detection unit 5 can cause a transient statistically significant change in one or more monitored parameters the reflected radiation. When the embolus 43 passes through the location A in FIG. 3, the reflected radiation 45a detected by the optical detector 32a is transiently altered in comparison to the reflected radiation observed in the absence of an embolus. Similarly, when the embolus passes through the location B in FIG. 3, the reflected radiation 45b detected by the optical detector 32b is transiently altered in comparison to the reflected radiation observed in the absence of an embolus. A significant change in the monitored parameter is detected by the detector 32b with a time lag after detection of a significant change by the detector 32a. The time interval between detection of the embolus 43 at the detectors 32a and 32b is inversely proportional to the velocity of the embolus 43 in the blood vessel 42. A statistically significant change in the intensity of the reflected light may be determined as follows. An average and standard deviation of the reflected radiation detected by a detector may be determined over a time window of predetermined length. A statistically significant change may be, for example, a change having a magnitude exceeding a predetermined factor times the determined standard deviation. The average and standard deviation may be periodically updated.

The detectors 32a and 32b generate a signal 40a and 40b (FIG. 2), respectively, indicative of the intensity of the reflected radiation detected by the detector. The signals 40a and 40b are input to the local processing unit 4 and\or 6 of the detection unit 5 or the control unit 20, respectively, which analyzes the signals for statistically significant transient changes indicative of the passing of an embolus in the FOV of the detection unit 5. When a statistically significant transient change is detected in the signal 40a is followed sequentially in time by a statistically significant transient change in the signal 40b, the processing unit 4 and\or 6 determine that an embolus has passed in the blood vessel 42 in the FOV of the detection unit 5. The determination that an embolus has been detected, may be confirmed by analyzing signals generated by other optical detectors, located in the same optical assembly or in one or more additional optical assemblies such as the optical detectors 32c and 32d, which are positioned lateral to the emitter 31. Alternatively or additionally, the determination may be confirmed by simultaneous detection of a transient change in local body impedance by the impedance detector 14 or by detection of a transient change in the spectroscopic properties of the blood. When the local processing unit 4 or 6 determines that an embolus has been detected, the local processing unit 4 or 6 sends a signal over the channel 7 to the processing unit 6 of the control unit 20 indicating that an embolus has been detected in the FOV of the detector 5. In response, the processing unit 6 activates the alarm 13. The processing unit 6 may also activate the transmitter 15 to transmit a message to a remote location indicating that an embolus has been detected. The transceiver 15 may also be used for data or software updates.

In another embodiment of the invention, the system 2 comprises two or more detection units 5 that can be positioned at different locations on the skin surface overlying one or more blood vessels (to allow simultaneous monitoring of two or more blood vessels).

1-21. (canceled)

22. A device for detecting a moving embolism in a first blood vessel comprising:
(a) one or more optical assemblies, each optical assembly comprising:
(i) a source of electromagnetic radiation adapted to deliver electromagnetic radiation to the first blood vessel;
(ii) a first optical detector detecting electromagnetic radiation produced by the source of electromagnetic radiation after the electromagnetic radiation has interacted with blood flowing in the first blood vessel;
(iii) a second optical detector detecting electromagnetic radiation produced by the source of electromagnetic radiation after the electromagnetic radiation has interacted with blood flowing in the first blood vessel, the source of electromagnetic radiation being positioned between the first and second optical detectors;
(b) a processor configured to perform the following steps:
(i) monitor one or more parameters of the electromagnetic radiation detected by the first optical detector and monitor the one or more parameters of the electromagnetic radiation detected by the second optical detector; and
(ii) detect a first predetermined or statistically significant transient change in one or more of the monitored parameters of the electromagnetic radiation detected by the first optical detector, and detect a second predetermined or statistically significant transient change in one or more of the monitored parameters of the electromagnetic radiation detected by the second optical detector, the second transient change occurring with a time lag after the occurrence of the first transient change; and
(iii) generate a sensible signal when a first predetermined or statistically significant transient change in one or more of the monitored parameters of the electromagnetic radiation is detected by the first optical detector, and a second predetermined or statistically significant transient change in one or more of the monitored parameters of the electromagnetic radiation is detected by the second optical detector, the second transient change occurring with a time lag after the occurrence of the first transient change;
wherein the optical assembly is configured to be immobilized at a location with the first and second optical detectors positioned along a longitudinal axis of the first blood vessel.

23. The device according to claim 22 wherein the source of electromagnetic radiation produces light having different wavelengths.

24. The device according to claim 22 wherein a monitored parameter is an intensity of the light.

25. The device according to claim 22 further comprising an alarm and the sensible signal is a signal produced by the alarm.

26. The device according to claim 22 further comprising a transceiver for communicating with a remote location.

27. The device according claim 22 wherein the one or more optical assemblies are located in one or more detection units each detection unit being configured to be immobilized at a location selected from (a) a skin surface over the first blood vessel, (b) a surface on the first blood vessel, or (c) a location under the skin surface over the first blood vessel.

28. The device according to claim 22 wherein one or more optical assemblies further comprise one or more additional optical detectors and the processor is further configured to monitor one or more parameters of radiation detected by the one or more additional optical detectors.

29. The device according to claim 22 wherein the step of detecting an embolus moving in the blood vessel further comprises detecting a transient change in one or more of the parameters of radiation detected by one or more of the additional optical detectors.

30. The device according to claim 29 comprising a third optical detector and a fourth optical detector, the third and fourth optical detectors being positioned on a line transverse to a line between the first and second detectors.

31. The device according to claim 22 wherein the optical detectors are arranged in a matrix.

32. The device according to claim 22 further comprising an impedance detector and the step of detecting an embolus moving in the blood vessel further comprises detecting a transient change in local body impedance.

33. The device according claim 22 further comprising one or more additional detection units each detection unit being configured to be immobilized at a location selected from (a) a skin surface over a second blood vessel, (b) a surface on a second blood vessel, or (c) a location under the skin surface over a second blood vessel.

34. The device according to claim 33 wherein the second blood vessel is the same as the first blood vessel.

35. The device according to claim 33 wherein the second blood vessel is different from the first blood vessel.

36. The device according to claim 33 wherein the first optical detector and the second optical detector are located in different optical assemblies.

37. The device according to claim 22 wherein a predetermined threshold is periodically updated.

38. The device according to claim 22 wherein the step of detecting an embolus moving in the blood vessel further comprises detecting a transient change in a spectral property of the blood.

39. The device according to claim 22 wherein the first blood vessel is a carotid artery.

40. The device according to claim 22 wherein two or more of the optical detectors are configured to detect reflected electromagnetic radiation.

41. A method for detecting a moving embolism in a first blood vessel comprising:
a. delivering electromagnetic radiation from a source of electromagnetic radiation to the first blood vessel;
b. detecting, by means of a first optical detector, electromagnetic radiation produced by the source of electromagnetic radiation after the electromagnetic radiation has interacted with blood flowing in the first blood vessel;
c. detecting, by means of a second optical detector, electromagnetic radiation produced by the source of electromagnetic radiation after the electromagnetic radiation has interacted with blood flowing in the first blood vessel, the source of electromagnetic radiation being positioned between the first and second optical detectors; and the first and second optical detectors are positioned along a longitudinal axis of the first blood vessel.
d. monitoring one or more parameters of the electromagnetic radiation detected by the first optical detector and monitoring the one or more parameters of the electromagnetic radiation detected by the second optical detector;
e. detecting a first predetermined or statistically significant transient change in one or more of the monitored parameters of the electromagnetic radiation detected by the first optical detector, and detecting a second predetermined or statistically significant transient change in one or more of the monitored parameters of the electromagnetic radiation detected by the second optical detector, the second transient change occurring with a time lag after the occurrence of the first transient change; and
f. activating an alarm when a first predetermined or statistically significant transient change in one or more of the monitored parameters of the electromagnetic radiation is detected by the first optical detector, and a second predetermined or statistically significant transient change in one or more of the monitored parameters of the electromagnetic radiation is detected by the second optical detector, the second transient change occurring with a time lag after the occurrence of the first transient change.
